(19) United States
(12) Patent Application Publication
(10) Pub. No.: US 2018/0360665 A1
(43) Pub. Date: Dec. 20, 2018

(54) DISPOSABLE WOUND BANDAGE WITH ADHESIVE FOR ADMINISTERING LIGHT THERAPY

(71) Applicant: InLightened Partners IP, LLC, Addison, TX (US)

(72) Inventor: David MELONE, Angel Fire, NM (US)

(73) Assignee: InLightened Partners IP, LLC, Addison, TX (US)

(21) Appl. No.: 16/009,713

(22) Filed: Jun. 15, 2018

Related U.S. Application Data
(60) Provisional application No. 62/520,044, filed on Jun. 15, 2017.

(51) Int. Cl. A61F 13/00; A61F 13/02; A61N 5/06

(52) U.S. Cl. CPC A61F 13/00; A61F 13/02; A61N 5/0613; A61F 2013/00655; A61F 2013/00863; A61B 2017/0023; A61N 2005/0645; A61N 2005/0652; A61N 2005/0659; A61N 2005/0663; A61N 2005/0629; A61N 2005/0632

(57) ABSTRACT

A wound bandage for administering light therapy is provided. The wound bandage may comprise a coated disposable flexible circuit. The disposable flexible circuit may comprise a set of plastic light-emitting diodes (LEDs). The disposable flexible circuit may be perforated and the set of LEDs may comprise at least one LED configured to emit light with a wavelength. The wound bandage may comprise a medical-grade adhesive disposed on a surface of the wound bandage. The adhesive may be configured to attach the wound bandage to a user's skin and maintain the wound bandage in a desired area. The wound bandage may comprise a socket configured to receive a connector coupled to an external control unit. The socket may transmit a control signal from the control unit to the set of LEDs and the control signal may pulse the set of LEDs at a frequency and a duty cycle.

### PRIORITY CLAIM

This application claims priority from U.S. Provisional Patent Application No. 62/520,044, filed on Jun. 15, 2017, which is hereby incorporated by reference in its entirety in the present application.

### TECHNICAL FIELD

The present disclosure relates generally to a disposable wound bandage with an adhesive for administering light therapy.

### BACKGROUND

Many patients suffer injuries to their tissue known as wounds. Varieties of wounds include abrasions, lacerations, punctures, incisions, ulcers, contusions, and avulsions. While the human body has natural biological wound-healing processes, light therapy may be used to accelerate these processes. Further, some patients (e.g., some patients with diabetes) may be unable to naturally complete a wound-healing process. Light therapy may be used to facilitate wound healing in such patients. Light therapy involves using a light-therapy system to administer light to the wound of a patient to accelerate or otherwise facilitate wound healing. Current systems, however, are expensive to produce and do not sufficiently accelerate wound healing. Because of their high cost, current systems are reused multiple times and require cleaning and/or sterilization after a certain number of uses or when being used by a new patient. Further, current systems are difficult to administer to a patient's wound because they include inconvenient attachment mechanisms and insufficient moisture control. Current systems also require an in-person assessment of the wound by a health-care provider in order to instruct the patient on a light-therapy treatment regimen (or “treatment program”) particular to his or her wound. Another disadvantage of current systems is the homogeneity of their shapes and sizes; different patients with different wounds may require light-treatment systems of different shapes and sizes.

The disclosed systems and methods are directed to overcoming one or more of the problems set forth above and/or other problems or shortcomings in the prior art.

### SUMMARY

In one aspect, the present disclosure is directed to a wound bandage for administering light therapy. The wound bandage may include a coated disposable flexible circuit comprising a first set of plastic light-emitting diodes (LEDs) and a second set of plastic LEDs. The disposable flexible circuit may be perforated. The first set of LEDs may comprise at least one LED configured to emit light with a first wavelength, and the second set of LEDs may comprise at least one LED configured to emit light with a second wavelength. The wound bandage may include a medical-grade adhesive disposed on a first surface of the wound bandage. The adhesive may be configured to attach the wound bandage to a user's skin and maintain the wound bandage is a desired area. The wound bandage may include a socket configured to receive a connector coupled to an external control unit. The socket may transmit a control signal from the control unit to the first and second set of LEDs. The control signal may pulse the first set of LEDs at a first frequency and a first duty cycle during a treatment program. The control signal may pulse the second set of LEDs at a second frequency and a second duty cycle during the treatment program.

In another aspect, the present disclosure is directed to a wound bandage for administering light therapy including a coated disposable flexible circuit comprising a set of plastic light-emitting diodes (LEDs). The disposable flexible circuit may be perforated and the set of LEDs may comprise at least one LED configured to emit light with a wavelength. The wound bandage may include a medical-grade adhesive disposed on a first surface of the wound bandage. The adhesive may be configured to attach the wound bandage to a user's skin and maintain the wound bandage in a desired area. The wound bandage may include a socket configured to receive a connector coupled to an external control unit. The socket may transmit a control signal from the control unit to the set of LEDs. The control signal may pulse the set of LEDs at a frequency and a duty cycle.

It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in to and constitute a part of this specification, illustrate the disclosed embodiments and, together with the description, serve to explain the principles of the various aspects of the disclosed embodiments. In the drawings:

FIG. 1 is a diagram of an exemplary system environment within which an exemplary wound bandage may operate;

FIG. 2 is an exploded view diagram of an exemplary wound bandage

FIG. 3 is an exploded view diagram of an exemplary wound bandage;

FIG. 4 is a bottom perspective view of an exemplary wound bandage;

FIG. 5 is a diagram of an exemplary environment within which an exemplary wound bandage may operate;

FIGS. 6A-6D are top views of exemplary wound bandages;

FIG. 7 is a top view of an exemplary wound bandage;

FIG. 8 is a bottom perspective view of an exemplary wound bandage;

FIGS. 9-12 are exploded views of exemplary wound bandages;

FIGS. 13-14 are diagrams of exemplary smartphones;

FIGS. 15A-15C are side views of an exemplary wound bandage;

FIG. 16 is a diagram of an exemplary environment within which an exemplary wound bandage may operate; and

FIGS. 17-18 are top perspective views of exemplary wound bandages.

It is to be understood that both the foregoing general descriptions and the following detailed descriptions are exemplary and explanatory only and are not restrictive of the claims.

### DESCRIPTION OF EXEMPLARY EMBODIMENTS

The present disclosure describes a disposable wound bandage with an adhesive for administering light therapy. The wound bandage is sufficiently flexible to serve as a bandage and sufficiently inexpensive that it may be disposed after a predetermined number of treatment sessions. The wound bandage administers treatment programs that optimally accelerate wound healing. The wound bandage has an adhesive for attaching the wound bandage to a patient's skin and maintaining the wound bandage in a desired area (e.g., over the patient's wound).

In some embodiments, the patient may use a software application to input information about their wound into a device and receive a treatment file specifying a treatment program for the patient. This input of information may occur at multiple points during the treatment program and may be used to adjust the treatment program during its execution. In some embodiments, the patient may use a software application to input information about their wound and generate a 3D-printing file. The patient, a healthcare provider, or a third party may use the 3D-printing file to print a wound bandage of a size and shape specific to the patient's needs in a 3D printer.

Reference will now be made to certain embodiments consistent with the present disclosure, examples of which are illustrated in the accompanying drawings. Wherever possible, the same reference numbers will be used throughout the drawings to refer to same or like parts.

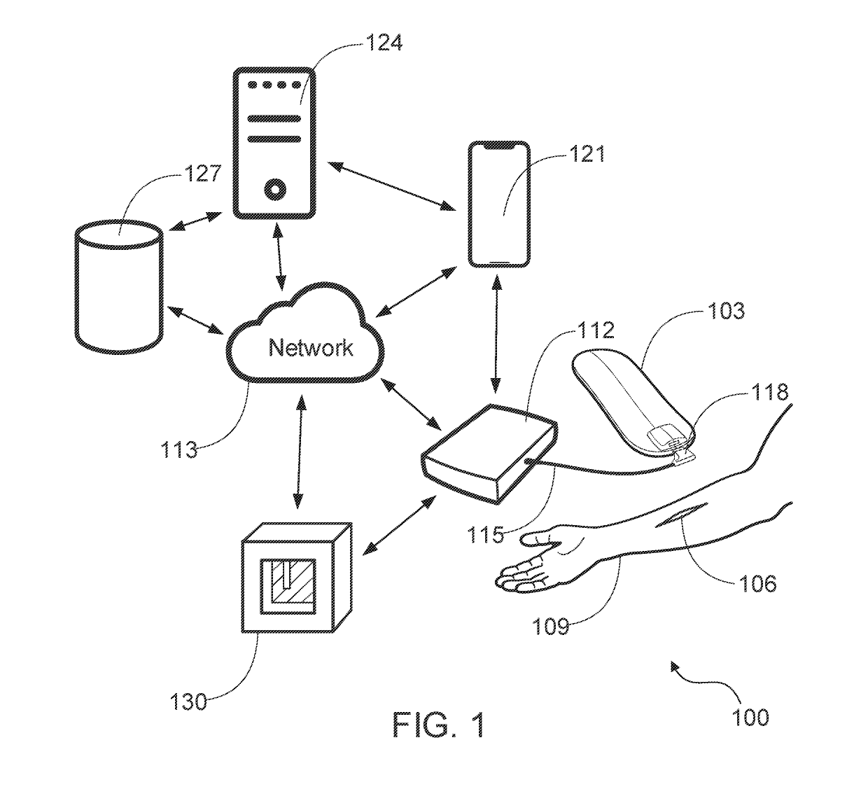

FIG. 1 is a diagram of an exemplary system environment 100 within which an exemplary wound bandage 103 may operate. Wound bandage 103 provides light therapy to a wound 106 on a part of a user's body, such as an arm 109.

Wound bandage 103 may be connected to a control unit 112 via a cable 115 with a connector 118. Control unit 112 may be capable of communicating with another device. For example, control unit 112 may communicate with an external smartphone 121, a server 124, a database 127, and/or a 3D printer 130. In some embodiments, control unit 112 and smartphone 121 may be the same device. In some embodiments, smartphone 121 may be another type of user device, such as a tablet or personal computer. Control unit 112 may be able to transmit or receive data over network 113. Network 113 may be implemented as, for example, the Internet, a wired Wide Area Network (WAN), a wired Local Area Network (LAN), a wireless LAN (e.g., Institute of Electrical and Electronics Engineers (IEEE) 802.11, Bluetooth, etc.), a wireless WAN (e.g., Worldwide Interoperability for Microwave Access (WiMAX)), a public switched telephone network (PSTN), an Integrated Services Digital Network (ISDN), an infrared (IR) link, a radio link, such as a Universal Mobile Telecommunications System (UMTS), Global System for Mobile Communications (GSM), Code Division Multiple Access (CDMA), broadcast radio network, cable television network, a satellite link, or the like. Network 113, in some embodiments, may comprise a plurality of interconnected wired or wireless data networks that receive data from one device (e.g., smartphone 121) and send it to another device (e.g., control unit 112).

In some embodiments, a user may input information about their wound into an application on smartphone 121. Smartphone 121 may use the inputted information to generate a treatment file. The treatment file specifies how wound bandage 103 is to administer light therapy to wound 106. In some embodiments, smartphone 121 may transmit the inputted information or data smartphone 121 extracts or generates from the inputted information to server 124, and server 124 may generate the treatment file. In some embodiments, smartphone 121 may transmit the inputted information or the data smartphone 121 extracts or generates from the inputted information to control unit 112, and control unit 112 may generate the treatment file.

The device generating the treatment file may do so by relying on a lookup table that specifies a relationship between the wound parameters (e.g., wound 106 type, wound 106 location, wound 106 size, wound 106 shape, the shape and size of the body part with wound 106, and/or wound 106 appearance) and optimal treatment program. For example, if a user inputs information into an application on smartphone 121 specifying that wound 106 is a puncture wound located on his or her arm 109, the device generating the treatment file may locate the optimal treatment program for a puncture wound on an arm in the lookup table. The device may generate the treatment file based on the located optimal treatment program. The lookup table may be stored on the device generating the treatment file. In some embodiments, the lookup table may be stored on database 127.

In some embodiments, the user may take a photograph of wound 106 and upload it into the application on smartphone 121. Smartphone 121 or another device generating the treatment file may run an image-comparison tool to compare the uploaded photograph with stored images of wounds successfully treated with associated treatment programs. The device generating the treatment file may select the treatment program associated with the most-similar wound image and generate the treatment file based on the selected treatment program. The stored images of wounds may have metadata associated with them, such as wound parameters for the wounds in the images (e.g., type of wound, size of wound, depth of wound, duration of successful treatment program). In some embodiments, the selected treatment program may be modified based on other information inputted by the user. For example, if the photograph of wound 106 matches most with a stored image of a particular wound but the user designates that his or her wound is deeper than the wound in the stored image, the selected treatment program may be modified to prescribe more treatment than would be otherwise prescribed.

In embodiments where the treatment file is generated externally to control unit 112, the treatment file may be transmitted to control unit 112. Control unit may generate a control signal based on the treatment file. Control unit 112 transmits the control signal to wound bandage 103 over cable 115 and connector 118. The control signal is used by control unit 112 to control the operation of Light-Emitting Diodes (“LEDs”) or other light sources on wound bandage 103.

In some embodiments, the user may specify the size and/or shape of wound 106. Instead or in addition, the user may specify the size and/or shape of the body part with wound 106. The device generating the treatment file or another device may generate a 3D-printing file. 3D printer 130 may use the 3D-printing file to print wound bandage 103. The printed wound bandage 103 may thus be uniquely shaped and sized to optimally treat wound 106 and conform to the body part containing wound 106. In embodiments where the 3D-printing file is generated externally to 3D printer 130, the 3D-printing file may be transmitted to 3D printer 130. In some embodiments, the treatment file may contain both the treatment program and the 3D-printing data. In this case, the treatment file may be generated instead or in addition to the 3D-printing file.

For ease of discussion, FIG. 1 depicts only particular components being connected to network 113. In some embodiments, however, more or fewer components may be connected to network 113.

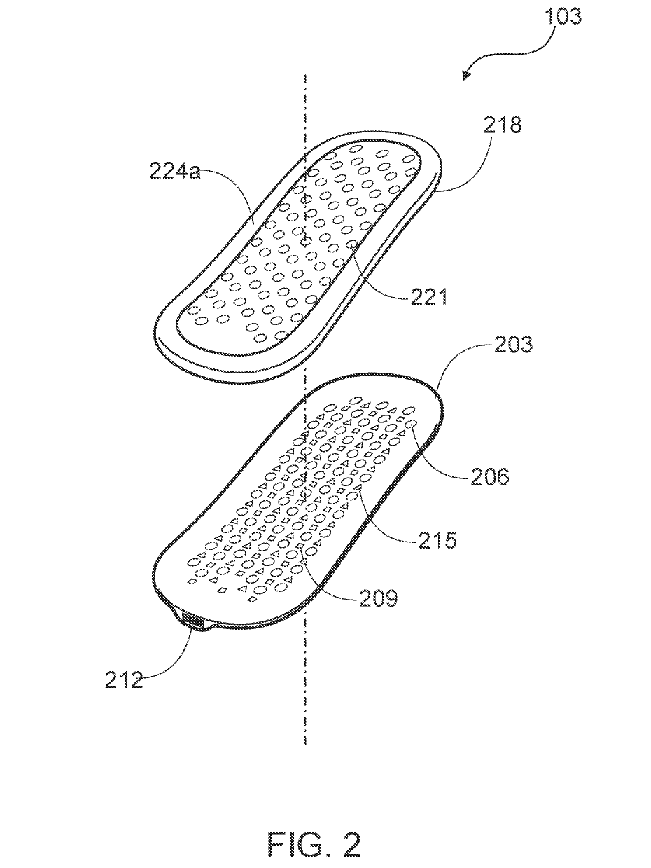

FIG. 2 is an exploded-view diagram of an exemplary wound bandage 103. Wound bandage 103 may comprise a flexible circuit 203. Flexible circuit 203 may be a flexible plastic substrate, such as polyimide, polyether ether ketone, or polyester (e.g., Mylar). Flexible circuit 203 may be disposable, RoHS compliant, and/or lead-free. In some embodiments, flexible circuit 203 may lack fiber glass. Flexible circuit 203 may be a single-layer or a multilayer circuit. Flexible circuit 203 may be perforated (i.e., may have perforations 206 indicated by circles).

Flexible circuit 203 comprises one or more LEDs or other light sources, such as LED 209. Flexible circuit 203 comprises a socket 212 configured to receive connector 118 and transmit a control signal from control unit 112 to the one or more LEDs. Connector 118 may be a plug suitable to transmit a control signal based on a treatment file from control unit 112. For example, connector 118 may be a Universal Serial Bus connector, and connector that transmits data using electromagnetic induction, an RJ45 connector, or another suitable connector.

LED 209 and/or other LEDs may be configured to emit light at one or more wavelengths. For example, LED 209 may be a monochromatic light emitting device, a substantially monochromatic LED, a single-color LED, an RGB LED, an infrared LED, and/or an ultraviolet LED. One or more LEDs in flexible circuit 203 may have a viewing angle greater than about 90 degrees, greater than about 120 degrees, greater than about 170 degrees, or about 180 degrees. A large viewing angle (e.g., greater than 150 degrees) may facilitate better administration of light to wound 106 from the LEDs and accelerate the wound-dealing process. One or more LEDs in flexible circuit 203 may be surface-mount devices. One or more LEDs in flexible circuit 203 may be plastic. One or more LEDs in flexible circuit 203 may be flat or rounded. Multiple LEDs may be controlled with the control signal simultaneously. LEDs made to operate in the same or similar manner by the control signal may form a set of LEDs. For example, LEDs the emit light at the same wavelength or wavelengths may form a set of LEDs. Instead or in addition, LEDs that are pulsed at the same frequency may form a set of LEDs.

In some embodiments, LED 209 and/or other LEDs in flexible circuit 203 may receive a control signal to emit light with one or more wavelengths. In some embodiments, the one or more LEDs may emit light with multiple wavelengths simultaneously (e.g., if the one or more LEDs are RGB LEDs). The treatment file may specify which one or more wavelengths of light to emit. The treatment file may specify the power, intensity, or luminous intensity with which one or more LEDs should emit one or more wavelengths of light and/or the voltage or current to supply to the LEDs. The treatment file may specify that the wavelength of the light emitted should be varied during the treatment program. For example, the treatment file may specify that the wavelength of the light emitted from wound bandage 103 should be varied during a single treatment session. Instead or in addition, the treatment file may specify that the wavelength of the light emitted from wound bandage 103 should be varied between different treatment sessions. In some embodiments, the wavelength of light emitted by one or more LEDs may be from about 430 nm to about 1,000 nm. The wavelength of light emitted by one or more LEDs may be from about 430 nm to about 950. The wavelength of light emitted by one or more LEDs may be from 850 nm to about 900 nm.

In some embodiments, flexible circuit 203 may comprise at least two sets of LEDs. A first set of LEDs may be activated when they receive a control signal to emit light with a first wavelength. A second set of LEDs may be activated when they receive a control signal to emit light with a second wavelength. For example, LEDs illustrated as squares, such as LED 209, may be the first set of LEDs, and LEDs illustrated as triangles, such as LED 215, may be the second set of LEDs. For example, one or more LEDs (e.g., LED 209) may emit light with a wavelength of about 880 nm and one or more LEDs (e.g., LED 215) may emit light with a wavelength of about 640 nm. In this example, the light at these two wavelengths may be emitted simultaneously or sequentially. The second set of LEDs may be activated before, during, or after the first set of LEDs cease to be activated. The control signal may vary the wavelength of light emitted from wound bandage 103 by activating LEDs emitting the first and/or the second wavelength during the treatment program. This may involve first activating LEDs that emit light at a first wavelength, (the first set of LEDs) ceasing to activate LEDs that emit light at the first wavelength (the first set of LEDs), and then activating LEDs that emit light at the second wavelength (the second set of LEDs). It is to be understood that the first and/or the second wavelength may be multiple wavelengths (e.g., the first wavelength may be three different wavelengths emitted by an RGB LED). It is to also be understood that, in some embodiments, the first and second wavelength may be the same wavelength. The treatment file may specify the power, intensity, or luminous intensity with which one or more LEDs should emit one or more wavelengths of light (e.g., to control the power with which light of different wavelengths is outputted by one or more RGB LEDs). The first set of LEDs and the second set of LEDs may be activated with the control signal simultaneously or in sequence. For example, the control signal may activate the first set of LEDs, cease activation of the first set of LEDs after a fixed period of time (e.g., about 20 seconds), and then activate the second set of LEDs. It is to be understood that, in some embodiments, flexible circuit 203 may comprise more than two sets of LEDs, such as three sets of LEDs. In this example, the first, second, and third sets of LEDs may be configured to emit light with a first, second, and third wavelength, respectively.

In some embodiments, the control signal may activate one or more LEDs by pulsing them. The LEDs may be pulsed at a frequency (the number of times they are turned on and off per second) and with a duty cycle (the percentage of a single on-off cycle the LED is powered on during the on-off cycle). For example, LED 209 may be pulsed at about 292 Hz with an about 50% duty cycle (i.e., 100 times per second and having the LED on for about 50% of each on-off cycle). In another non-limiting example, LED 209 or another LED may be pulsed at about 4,672 Hz with an about 50% duty cycle. Instead of in addition, LED 209 or another LED may be pulsed with an about 75% duty cycle. LED 209 may be pulsed with sufficient voltage and/or current to generate, for example, about 40 millicandelas. The treatment file may specify that the pulsing frequency of one or more LEDs should be varied during a single treatment session. Instead or in addition, the treatment file may specify that the pulsing frequency of one or more LEDs should be varied between different treatment sessions. The treatment file may specify that the duty cycle of one or more LEDs should be varied during a single treatment session. Instead or in addition, the treatment file may specify that the duty cycle of one or more LEDs should be varied between different treatment sessions. The treatment file may specify that the luminous intensity or other parameter should be varied during a single treatment session. Instead or in addition, the treatment file may specify that the luminous intensity or other parameter should be varied between different treatment sessions. A first set of LEDs may be pulsed at a first frequency and a first duty cycle and a second set of LEDs may be pulsed at the first frequency and the first duty cycle. In some embodiments, the second set of LEDs may be pulsed at a second frequency and the first duty cycle. In some embodiments, the second set of LEDs may be pulsed at the first frequency and a second duty cycle. The second set of LEDs may be pulsed before, during, or after the second set of LEDs is pulsed. Different sets of LEDs may be pulsed to generate light with different luminous intensity or the same luminous intensity.

A single treatment session may last, for example, about 20 minutes or about 3 hours. In some embodiments, a treatment session may last from about 1 minute to about 30 minutes. A treatment session may last from about 10 minutes to about 30 minutes. A full treatment program may last, for example, about 6 months and be comprised of multiple treatment sessions. Wavelengths of light emitted by LEDs may include, for example, red light (about 640 nm wavelength), blue light (about 485 nm wavelength), and infrared (about 880 nm wavelength). LEDs may be pulsed sequentially at, for example, about 23 Hz, about 72 Hz, about 46 Hz, about 96 Hz, and about 1168 Hz. LEDs may be pulsed sequentially at, for example, about 292 Hz and about 4,672. In some embodiments, the pulsing frequency may be switched approximately every minute. By switching the pulsing frequency from, for example, about 292 Hz to about 4,672 Hz, the pulsing frequency may be considered to be varied from about 292 Hz to about 4,672 Hz during the treatment program. In some embodiments, the series of frequencies at which LEDs are pulsed may be repeated during a treatment session. In some embodiments, one or more LEDs may be pulsed at a frequency corresponding to a note on a musical scale and/or its harmonics. For example, LED 209 may be pulsed at about 82.407 Hz, corresponding to the musical note E₂, and/or harmonics of E₂. One or more LEDs may be pulsed at a frequency from about 1 Hz to about 10,000 Hz. One or more LEDs may be pulsed at a frequency from about 70 Hz to about 4,999 Hz. One or more LEDs may be pulsed at a frequency of about 292 Hz, about 584 Hz, about 1,168 Hz, about 2,336 Hz, about 4,672 Hz, about 73 Hz, and/or about 146 Hz. In some embodiments, the duty cycle may be about 25%, about 50%, or about 75%. The duty cycle may be from about 25% to about 75%.

Wound bandage 103 comprises a coating layer 218. Coating layer 218 may be an FDA approved and/or biomedical-grade coating, such as polypropylene, polyethylene, and/or or silicone. Coating layer 218 may protect devices on flexible circuit 203 from contact with moisture from the user's skin and protect wound 106 from being contaminated or infected with particles from devices on flexible circuit 203. Thus, flexible circuit 203 may be considered to be “coated” by coating layer 218. Coating layer 218 is perforated with perforations, such as perforation 221, indicated by circles. In some embodiments, perforations 206 and 221 may align with one another to promote through wound bandage 103. Coating layer 218 may be translucent or transparent to permit light from flexible circuit 203 to travel to wound 106. Coating layer 218 may be translucent or transparent to the specific wavelengths of light emitted by one or more LEDs.

Coating layer 218 may comprise an adhesive 224 disposed on at least one surface. Adhesive 224 may be a medical-grade adhesive, such as an acrylic, hydrogel, or silicone gel. The material for adhesive 224 may be selected such that adhesive 224 does not damage skin on arm 109 or another body part when wound bandage 103 is removed from arm 109. Adhesive 224 may be disposed about the perimeter of coating layer 218.

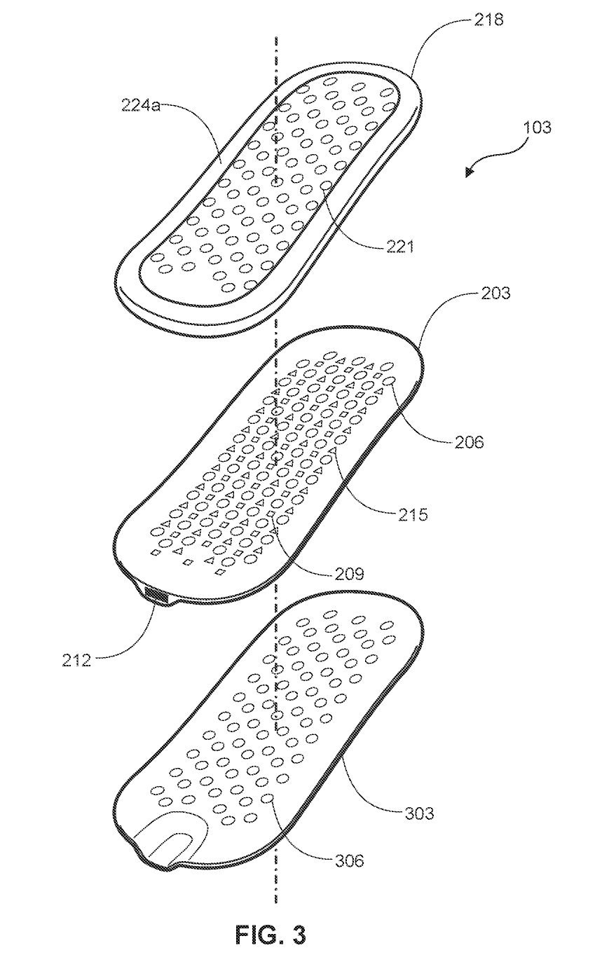

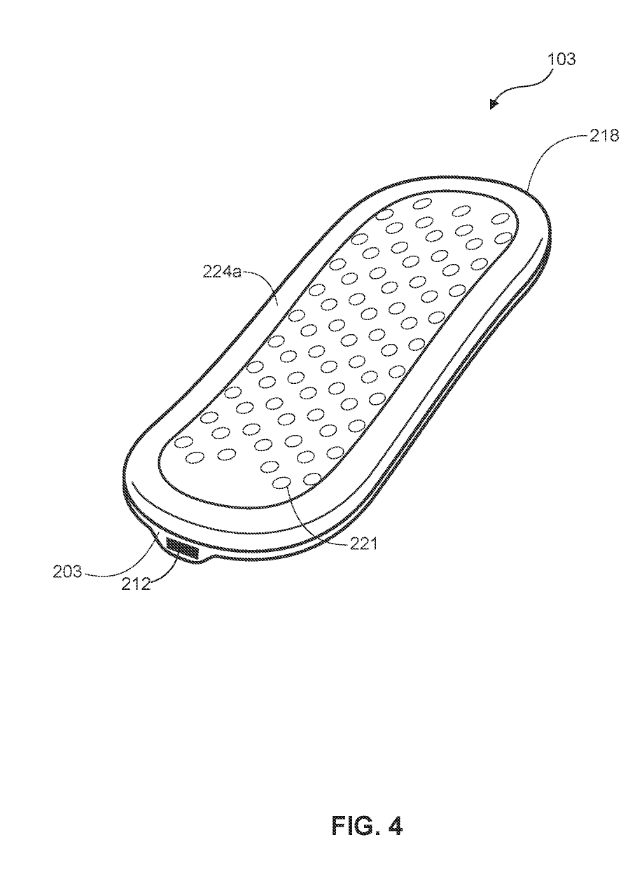

FIG. 3 is an exploded view diagram of an exemplary wound bandage 103 similar to wound bandage 103 of FIG. 2. Coating layer 218 of wound bandage 103 illustrated in FIG. 3 may have perforations 221 illustrated as circles. Flexible circuit 203 may have perforations 206 illustrated as circles. First set of LEDs 209 is illustrated with squares and the second set of LEDs 215 is illustrated with triangles. In some embodiments, wound bandage 103 may have an outer covering 303. Outer covering 303 may serve to prevent unwanted contact with flexible circuit 203. Outer covering 303 may be made of a mesh material (e.g., with a gauze weave pattern) or another material. Outer covering 303 may have different colors, prints, texture, and/or customizable graphics on its top side (not shown). Outer covering 303 may have perforations 306, illustrated as circles. In some embodiments, outer covering 303 may have no perforations. In some embodiments, any combination of perforations 221, 206, and 306 may align to promote airflow through wound bandage 103. FIG. 4 is a bottom perspective view of the wound bandage 103 illustrated in FIG. 3.

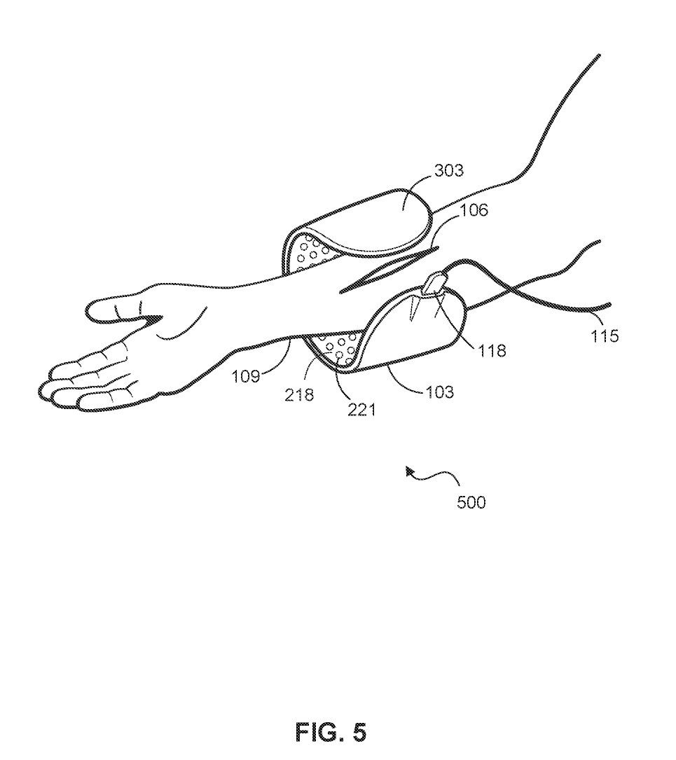

FIG. 5 is a diagram of an exemplary environment 500 within which wound bandage 103 may operate. While the exemplary wound bandage 103 illustrated in FIG. 5 has no perforations on outer covering 303, it is to be understood that some embodiments of wound bandage 103 may have perforations on outer covering 303. While the exemplary wound bandage 103 illustrated in FIG. 5 has no adhesive on coating layer 218, it is to be understood that some embodiments of wound bandage 103 may have an adhesive on coating layer 218.

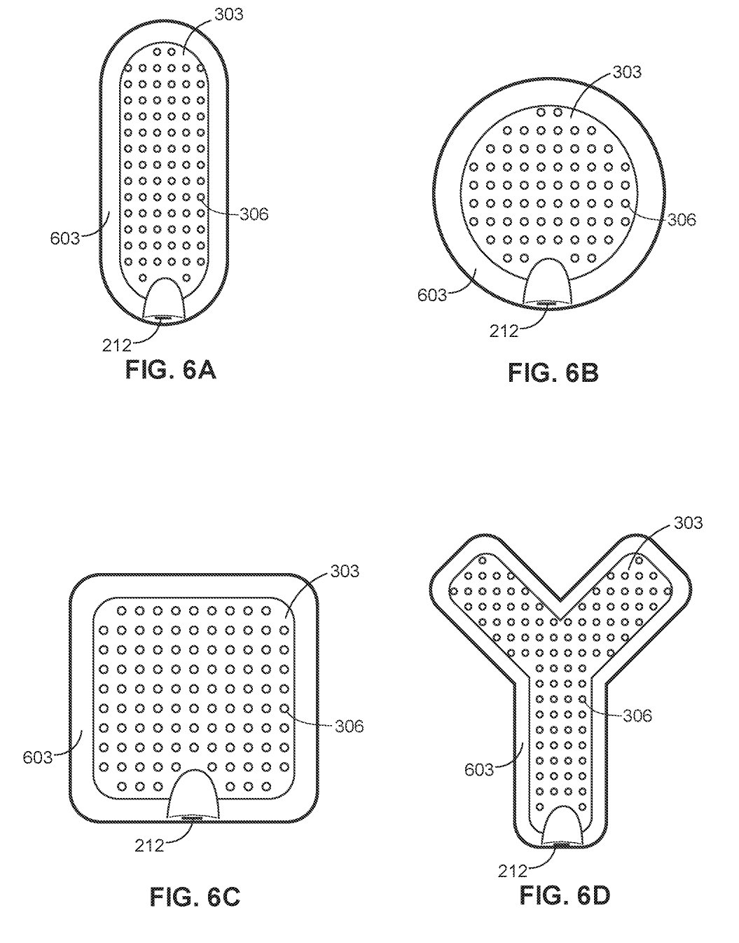

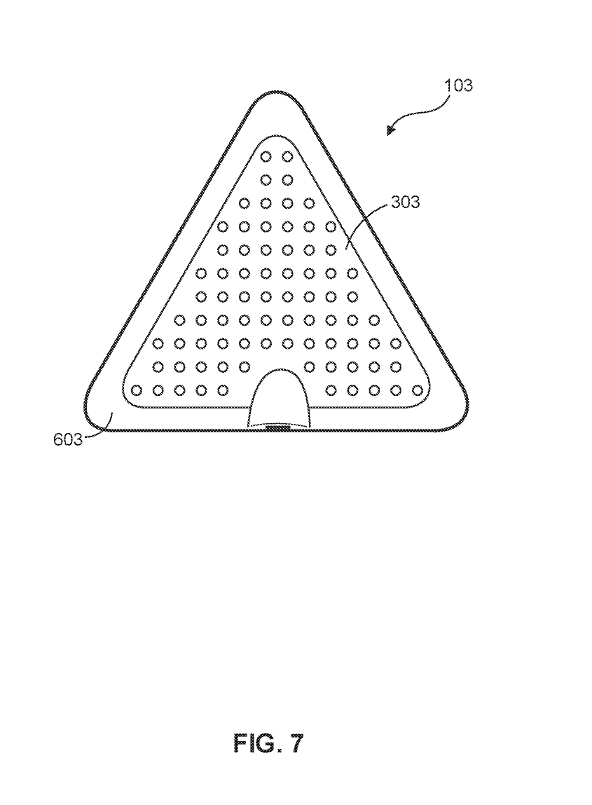

FIGS. 6A through 6D and 7 are top views of exemplary embodiments of wound bandages 103. These exemplary embodiments may have perforations 306 on outer covering 303. In some embodiments, wound-bandage shapes illustrated in FIGS. 6A through 6D and 7 may be used without perforations 306 on outer covering 303. In some embodiments, wound-bandage shapes illustrated in FIGS. 6A through 6D and 7 may have no outer covering 303. One or more exemplary wound bandages 103 illustrated in FIGS. 6A through 6D and 7 may have an outer adhesive 603 disposed along the perimeter of outer covering 303. In some embodiments, outer adhesive 603 may be disposed along only a portion of the perimeter of outer covering 303 or on another section of outer covering 303. Outer adhesive may keep wound bandage 103 securely in place if wound bandage 103 is wrapped around itself. In some embodiments, outer covering 303 may have no outer adhesive 603.

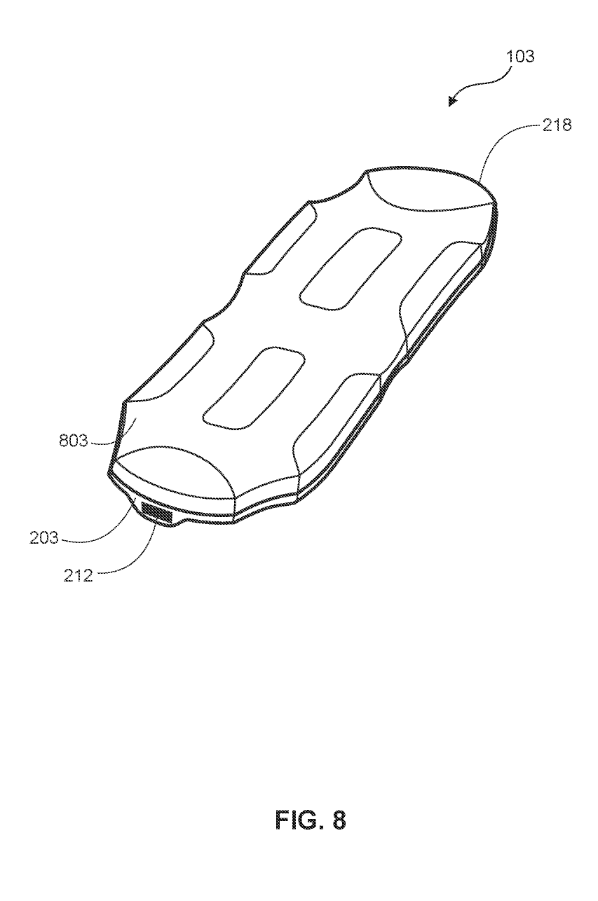

FIG. 8 is a bottom perspective view of an exemplary wound bandage 103. Coating layer 218 may comprise an air channel 803 instead of in addition to perforations (not shown). Air channel 803 may facilitate airflow through wound bandage 103. In some embodiments, air channel 803 may be thin enough to be placed between columns of LEDs. There may be one or more air channels 803 that are discrete or interconnected in coating layer 218.

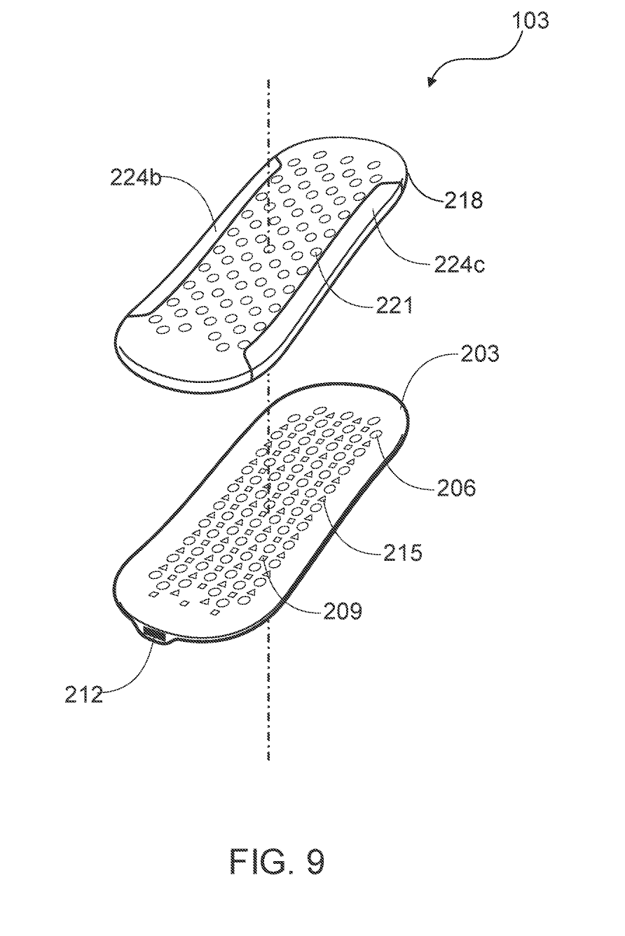

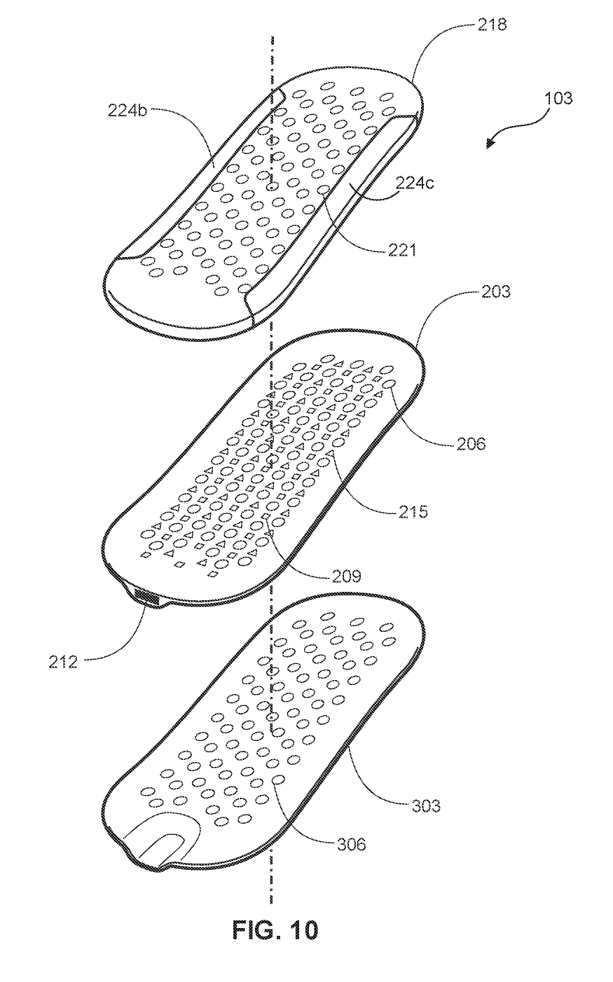

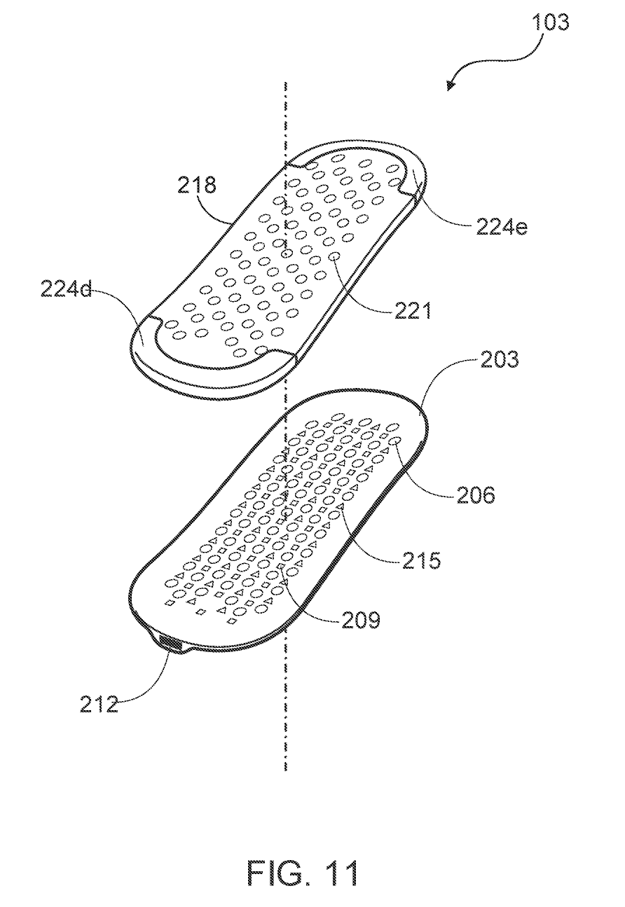

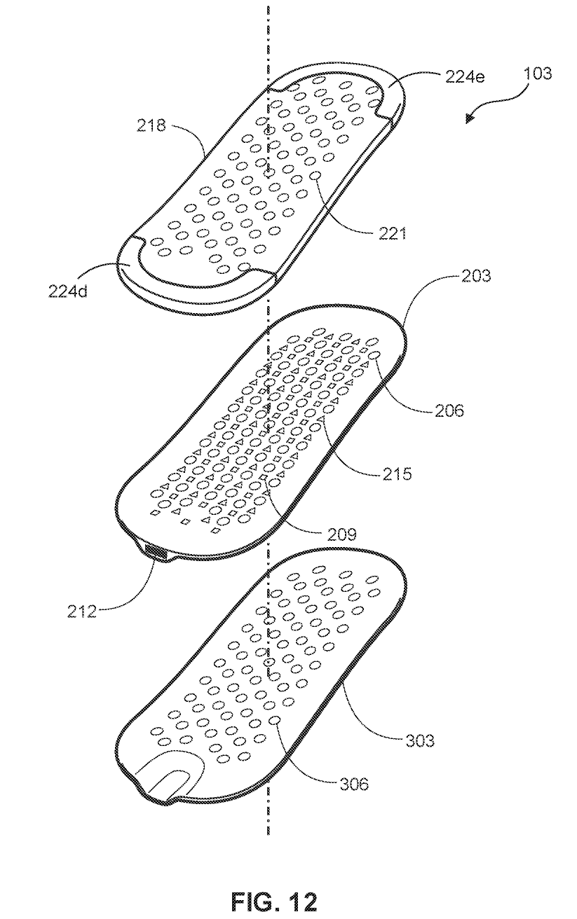

In some embodiments, adhesive 224 may be disposed on other potions of coating layer 218. For example, FIGS. 9 and 10, exploded-view diagrams of exemplary wound bandages 103, show adhesive 224b, 224c on the left and right edges of coating layer 218, respectively. FIGS. 11 and 12, exploded-view diagrams of exemplary wound bandages 103, show adhesive 224d, 224e on the proximal and distal edges of coating layer 218, respectively.

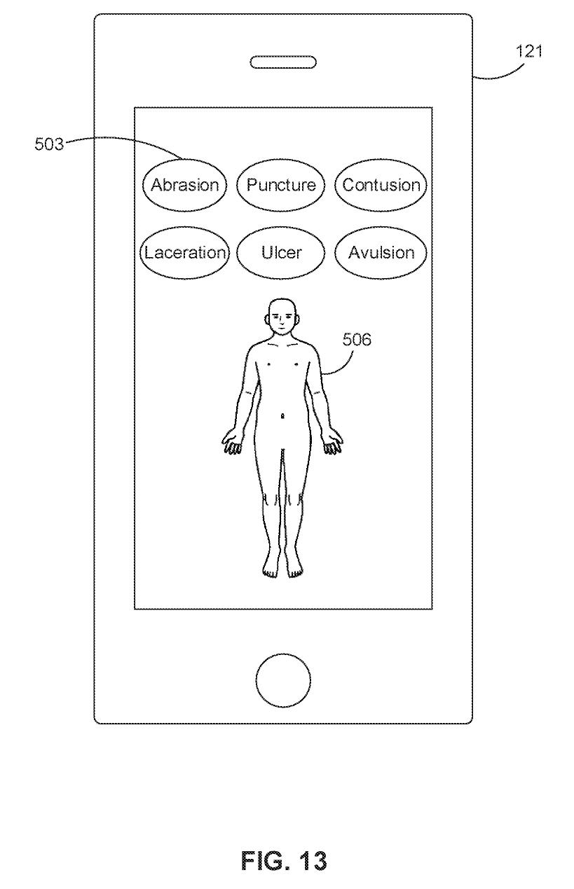

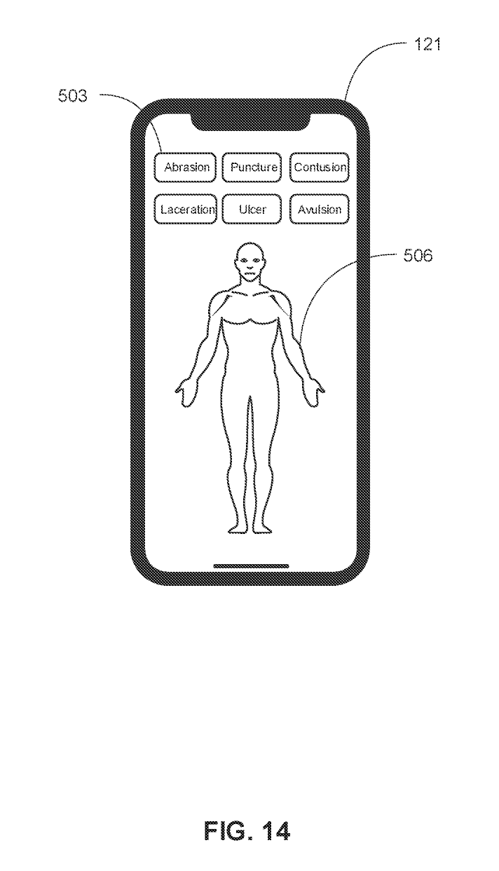

FIG. 13 is a diagram of an exemplary smartphone 121. In some embodiments, a user may use an application on smartphone 121 to select the type of wound they want to treat, including but not limited to an abrasion, laceration, puncture, incision, ulcer, contusion, or avulsion. The user may use the application on smartphone 121 to select the part of their body on which the wound appears. For example, the user may select icon 503 to indicate they seek to treat an abrasion and select the arm 506 on the displayed body to indicate that they seek to treat an abrasion on an arm. In some embodiments, a user may use smartphone 121 to select the cause of their wound (e.g., diabetes, jaundice, etc.). Smartphone 121 may use inputted information to locate an appropriate treatment file stored on smartphone 121, server 124, database 127, or control unit 112, and transmit the treatment file to control unit 112. In some embodiments, smartphone 121 may transmit the inputted information to server 124 and server 124 may locate an appropriate treatment file stored on smartphone 121, server 124, database 127, or control unit 112, and transmit the treatment file to control unit 112. Smartphone 121 may be used to initiate or cease execution of a treatment program by control unit 112. Instead or in addition, controls on control unit 112 may be used to initiate or cease execution of the treatment program by control unit 112. In some embodiments, the application on smartphone 121 may prompt the user answer questions or upload photographs pertaining to their treatment (e.g., the wound's appearance). The user feedback may be used to adjust the treatment program and/or the treatment file. For example, a photograph of wound 106 before treatment and a photograph of wound 106 during treatment may be compared to determine how many more treatment sessions are needed or how to adjust the treatment program's parameters (e.g., what wavelengths of light to use, what pulsing frequency to use, what duty to use, what power output to use). In some embodiments, smartphone 121 may be another type of computing device (e.g., a tablet or personal computer). FIG. 14 is another diagram of an exemplary embodiment of smartphone 121, icon 503, and arm 506.

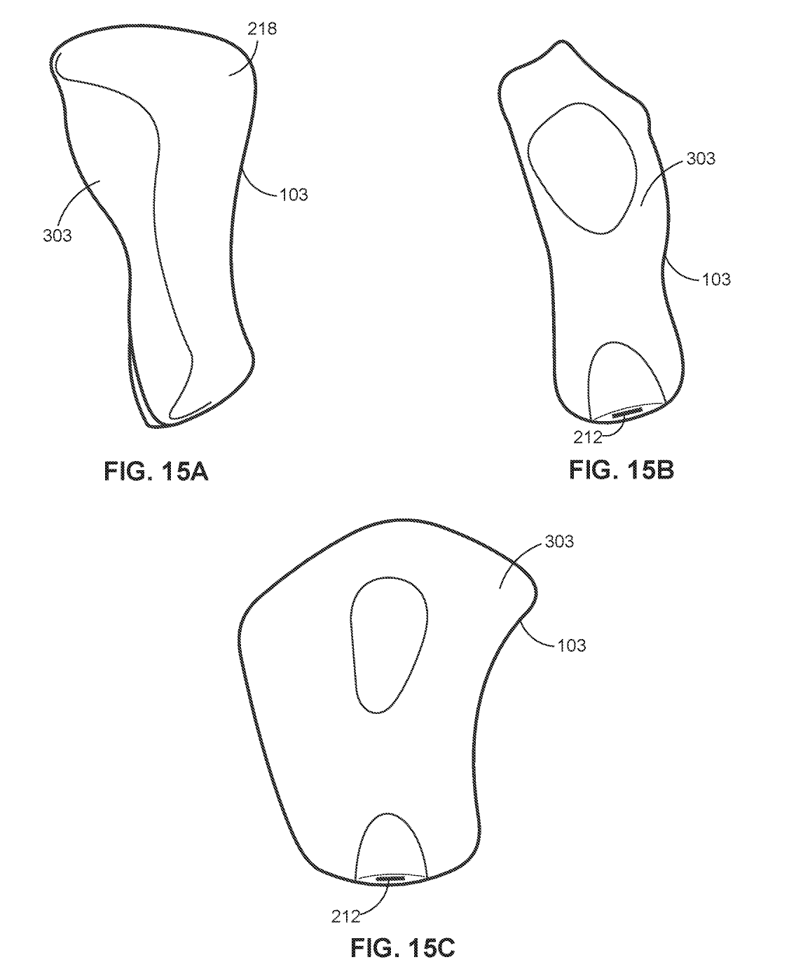

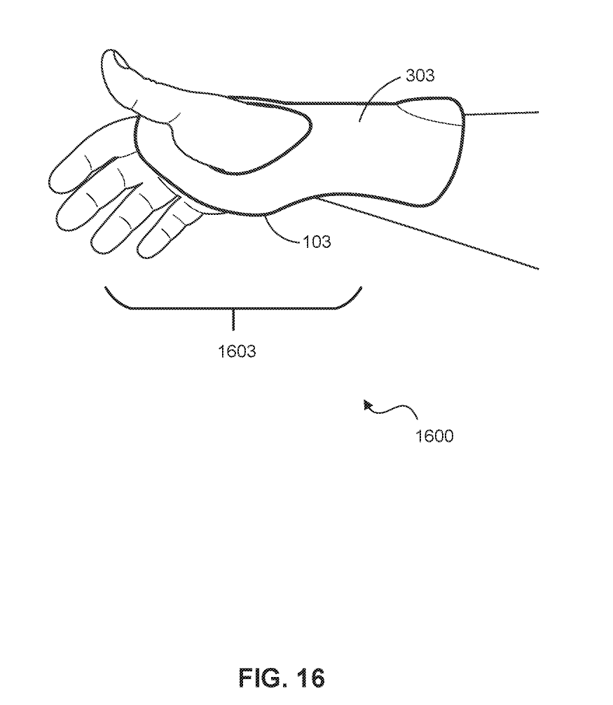

FIGS. 15A through 15C are side views of an exemplary wound bandage 103. Wound bandage 103 may be printed with 3D printer 130. Wound bandage 103 may be printed using data pertaining to the body part for which wound bandage 103 is intended. For example, the data may be an image of the body part (e.g., a hand and wrist) and/or a 3D scan of the body part. FIG. 16 is a diagram of an exemplary environment 1600 within which an exemplary wound bandage 103 may operate. For example, wound bandage 103 of FIGS. 15A through 15C may be worn on the user's hand and wrist 1603. While perforations are not illustrated in FIGS. 15A through 15C for clarity, it is to be understood that wound bandage 103 may have perforations on outer covering 303 and/or coating layer 218.

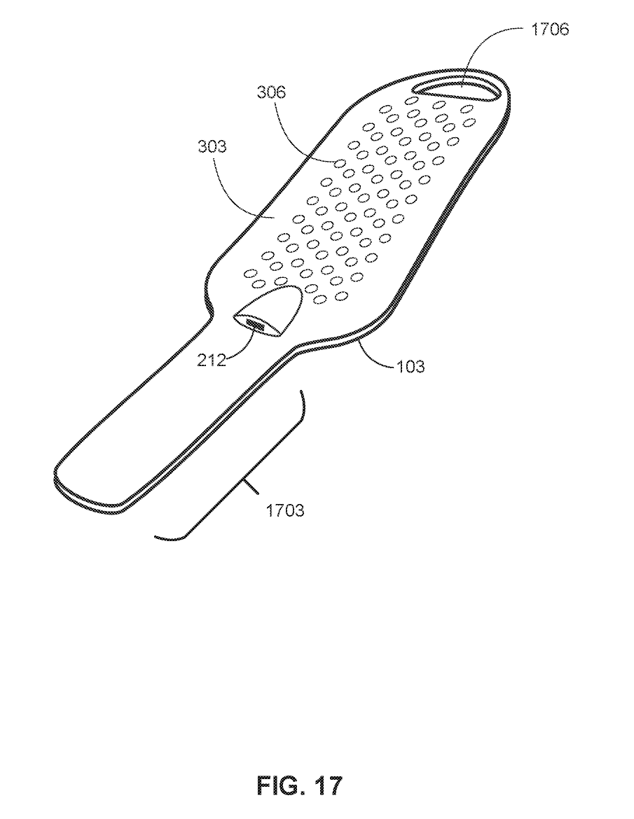

FIG. 17 is a top perspective view of an exemplary wound bandage 103. In an embodiment, wound bandage 103 may have a narrow portion 1703 that may be inserted into and wrapped over slot 1706 to secure wound bandage 103 in place.

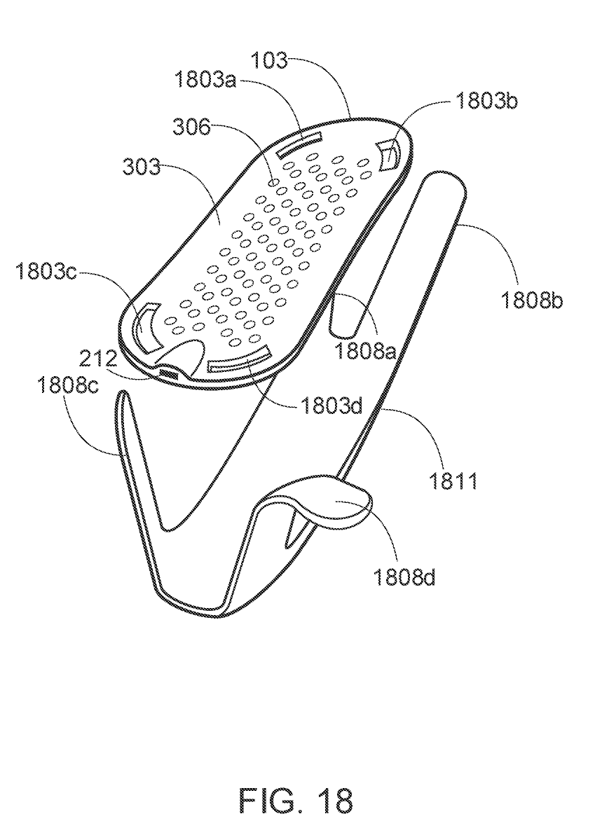

FIG. 18 is a top perspective view of an exemplary wound bandage 103. In an embodiment, wound bandage 103 may have one or more slots, such as slots 1803a, 1803b, 1803c, and 1803d, that may receive narrow portions 1808a, 1808b, 1808c, and 1808d, respectively, of attachment portion 1811. Narrow portions 1808a, 1808b, 1808c, and 1808d may be wrapped over slots 1808a, 1808b, 1808c, and 1808d, respectively to secure wound bandage 103 in place.

Wound bandage 103 may be disposable due to its low cost of production. Wound bandage 103 may have a low cost of production due to it having, in some embodiments, plastic LEDs (e.g., LEDs with plastic substrates) rather than glass LEDs. Wound bandage 103 may have a low cost of production because, in some embodiments, it is externally powered by control unit 112 and does not require the power source and corresponding circuitry to be manufactured for each wound bandage 103. Wound bandage 103 may have a low cost of production because, in some embodiments, it has only circuitry and/or devices necessary to transmit the control signal from control unit 112 to one or more LEDs; additional circuitry may increase the cost of production. Wound bandage 103 may have a low cost because, in some embodiments, its circuit is flexible due to not being made of fiberglass; using fiberglass may increase the cost of production. Wound bandage 103 may have a low cost because, in some embodiments, flexible circuit 203 is a single-layer circuit rather than a multilayer circuit; using a multilayer circuit may increase the cost of production.

Certain embodiments of the present disclosure can be implemented as software on a general-purpose computer or on another device.

The term “about” or “approximately” as used herein means within an acceptable error range for the particular value as determined by one of ordinary skill in the art, which will depend in part on how the value is measured or determined, e.g., the limitations of the measurements system. For example, “about” can mean within one or more than one standard deviation per the practice in the art. Alternatively, “about” can mean a range of up to 20%, such as up to 10%, up to 5%, and up to 1% of a given value.

It is to be understood that different styles of shading or shading patterns used in the figures may represent different colors, different styles of shading, or different shading patterns.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments.

The features and advantages of the disclosure are apparent from the detailed specification, and thus it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and, accordingly, all suitable modifications and equivalents falling within the scope of the disclosure may be resorted to.

Computer programs, program modules, and code based on the written description of this specification, such as those used by the microcontrollers, are readily within the purview of a software developer. The computer programs, program modules, or code can be created using a variety of programming techniques. For example, they can be designed in or by means of Java, C, C++, assembly language, or any such programming languages. One or more of such programs, modules, or code can be integrated into a device system or existing communications software. The programs, modules, or code can also be implemented or replicated as firmware or circuit logic.

Another aspect of the disclosure is directed to a non-transitory computer-readable medium storing instructions which, when executed, cause one or more processors to perform the methods of the disclosure. The computer-readable medium may include volatile or non-volatile, magnetic, semiconductor, tape, optical, removable, non-removable, or other types of computer-readable medium or computer-readable storage devices. For example, the computer-readable medium may be the storage unit or the memory module having the computer instructions stored thereon, as disclosed. In some embodiments, the computer-readable medium may be a disc or a flash drive having the computer instructions stored thereon.

Moreover, while illustrative embodiments have been described herein, the scope of any and all embodiments include equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

What is claimed is:

1. A wound bandage for administering light therapy comprising:
a coated disposable flexible circuit comprising a first set of plastic light-emitting diodes (LEDs) and a second set of plastic LEDs, wherein:
the disposable flexible circuit is perforated;
the first set of LEDs comprises at least one LED configured to emit light with a first wavelength, and
the second set of LEDs comprises at least one LED configured to emit light with a second wavelength;
a medical-grade adhesive disposed on a first surface of the wound bandage, wherein the adhesive is configured to attach the wound bandage to a user's skin and maintain the wound bandage is a desired area;
a socket configured to receive a connector coupled to an external control unit, wherein:
the socket transmits a control signal from the control unit to the first and second set of LEDs,
the control signal pulses the first set of LEDs at a first frequency and a first duty cycle during a treatment program, and
the control signal pulses the second set of LEDs at a second frequency and a second duty cycle during the treatment program.

2. The wound bandage of claim 1, wherein the disposable flexible circuit is lead-free.

3. The wound bandage of claim 1, wherein the coating prevents contact between the user's skin and devices in the disposable flexible circuit.

4. The wound bandage of claim 1, wherein at least one of the first set of LEDs or the second set of LEDs is a set of surface-mount LEDs with a viewing angle greater than 120 degrees.

5. The wound bandage of claim 1, wherein the control signal pulses the second set of LEDs after pulsing and ceasing to pulse the first set of LEDs.

6. The wound bandage of claim 1, wherein the control signal pulses the second set of LEDs after pulsing the first set of LEDs for about 20 seconds and ceasing to pulse the first set of LEDs.

7. The wound bandage of claim 1, wherein the first and second wavelength are about 880 nm and about 640 nm, respectively.

8. The wound bandage of claim 1, wherein the first frequency is varied during the treatment program.

9. The wound bandage of claim 1, wherein the first frequency is varied from about 292 Hz to about 4,672 Hz during the treatment program.

10. The wound bandage of claim 1, wherein the first duty cycle is varied during the treatment program.

11. The wound bandage of claim 1, wherein the first duty cycle is varied from about 50% to about 75% during the treatment program.

12. The wound bandage of claim 1, wherein at least one of the first and second frequencies or the first and second duty cycles are the same.

13. A wound bandage for administering light therapy comprising:
a coated disposable flexible circuit comprising a set of plastic light-emitting diodes (LEDs), wherein the disposable flexible circuit is perforated and the set of LEDs comprises at least one LED configured to emit light with a wavelength, and
a medical-grade adhesive disposed on a first surface of the wound bandage, wherein the adhesive is configured to attach the wound bandage to a user's skin and maintain the wound bandage in a desired area; and
a socket configured to receive a connector coupled to an external control unit, wherein the socket transmits a control signal from the control unit to the set of LEDs and the control signal pulses the set of LEDs at a frequency and a duty cycle.

14. The wound bandage of claim 13, wherein the coating prevents contact between the user's skin and devices in the disposable flexible circuit.

15. The wound bandage of claim 13, wherein the set of LEDs is a set of surface-mount LEDs with a viewing angle greater than 120 degrees.

16. The wound bandage of claim 13, wherein the wavelength is varied during the treatment program.

17. The wound bandage of claim 13, wherein the wavelength is varied from about 880 nm to about 640 nm during the treatment program.

18. The wound bandage of claim 13, wherein the frequency is varied during the treatment program.

19. The wound bandage of claim 13, wherein the frequency is varied from about 292 Hz to about 4,672 Hz during the treatment program.

20. The wound bandage of claim 13, wherein the duty cycle is varied during the treatment program.
